(19) United States
(12) Patent Application Publication
(10) Pub. No.: US 2022/0050752 A1
(43) Pub. Date: Feb. 17, 2022

(54) DATA STORAGE GEOGRAPHIC LOCATION COMPLIANCE AND MANAGEMENT

(71) Applicant: Seagate Technology LLC, Fremont, CA (US)

(72) Inventor: Basavaraj Kirunge, Pune (IN)

(21) Appl. No.: 16/993,641

(22) Filed: Aug. 14, 2020

(51) Int. Cl. G06F 11/14; G06F 16/27; G06Q 30/00; G06Q 50/26

(52) U.S. Cl. CPC G06F 11/1451; G06F 16/275; G16H 10/60; G06Q 50/265; G06F 2201/80; G06Q 30/0185

(57) ABSTRACT

A method includes automatically associating a dataset with a first tag indicating that the dataset is subject to a data compliance law, automatically associating the dataset with a second tag indicating a geographic location of a source of the dataset, selecting a remote backup destination for the dataset that is compliant by comparing the first tag and the second tag to a compliance policy, and transmitting a replica of the dataset to the remote backup destination.

### SUMMARY

In certain embodiments, method is disclosed. The method includes automatically associating a dataset with a first tag indicating that the dataset is subject to a data compliance law, automatically associating the dataset with a second tag indicating a geographic location of a source of the dataset, selecting a remote backup destination for the dataset that is compliant by comparing the first tag and the second tag to a compliance policy, and transmitting a replica of the dataset to the remote backup destination.

In certain embodiments, a non-transitory computer-readable medium stores instructions that, when executed by a processor, cause the processor to automatically associate a dataset with a first tag indicating that the dataset is subject to a data compliance law, automatically associate the dataset with a second tag indicating a geographic location of a source of the dataset, select a remote backup destination for the dataset that is compliant by comparing the first tag and the second tag to a compliance policy, and initiate transmission of a replica of the dataset to the remote backup destination.

In certain embodiments, a system including a first data storage system with a computer. The computer is configured to receive datasets from a data source and associate the datasets with location metadata indicating the geographic origins of the datasets, based on a location from an internet protocol address or a global positioning system. The computer is configured to scan the datasets, automatically determine that the dataset includes personally-identifiable information (PII) by comparing the scanned datasets to a library of data compliance indicators, and associate the datasets with compliance metadata indicating that the datasets include PII.

While multiple embodiments are disclosed, still other embodiments of the present invention will become apparent to those skilled in the art from the following detailed description, which shows and describes illustrative embodiments of the invention.

Accordingly, the drawings and detailed description are to be regarded as illustrative in nature and not restrictive.

### BRIEF DESCRIPTION OF THE DRAWINGS

FIG. 1 shows a schematic of a data ecosystem, in accordance with certain embodiments of the present disclosure.

FIG. 2 shows a block diagram of a data storage system, in accordance with certain embodiments of the present disclosure.

FIG. 3 shows a block diagram of a data structure, in accordance with certain embodiments of the present disclosure.

FIG. 4 shows a block diagram of another data storage system, in accordance with certain embodiments of the present disclosure.

FIG. 5 depicts a block diagram of steps of a data transfer method, in accordance with certain embodiments of the present disclosure.

FIG. 6 shows a block diagram of components of a computer for carrying out methods and functions described herein, in accordance with certain embodiments of the present disclosure.

While the disclosure is amenable to various modifications and alternative forms, specific embodiments have been shown by way of example in the drawings and are described in detail below. The intention, however, is not to limit the disclosure to the particular embodiments described but instead is intended to cover all modifications, equivalents, and alternatives falling within the scope of the appended claims.

### DETAILED DESCRIPTION

Many regions, countries, and states have their own data laws and regulations that limit how and where sensitive data (e.g., personal data, health data, certain technical data) is stored and transferred. For example, a country may limit the extent personal data is exported and stored outside the country. Complying with these laws and regulations is challenging. Certain embodiments of the present disclosure are accordingly directed to data storage systems that can help ensure compliance with data laws and regulations.

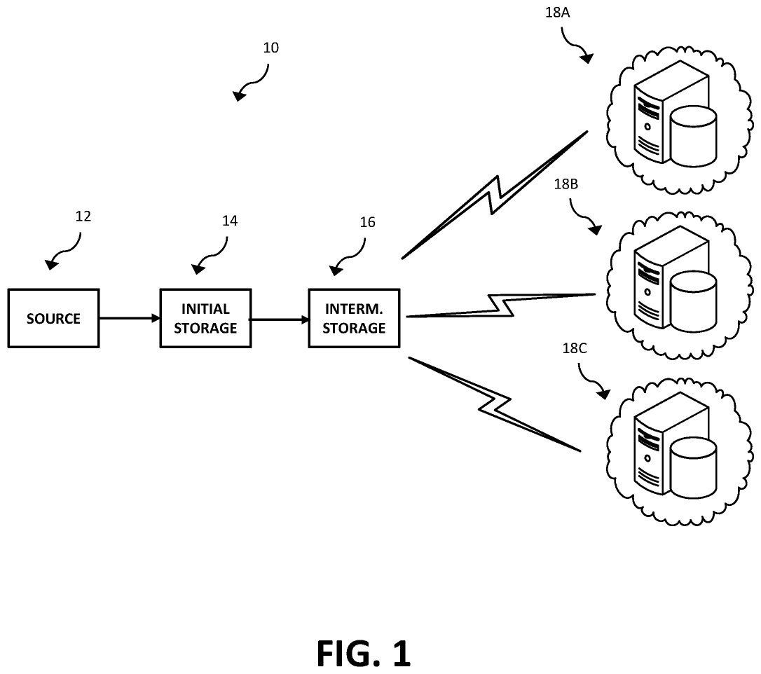

FIG. 1 shows a schematic of a data ecosystem 10 including one or more data sources 12, initial data storage systems 14, intermediate data storage systems 16, and backup data storage systems 18A-C In certain embodiments, the data ecosystem 10 represents a private enterprise's data ecosystem. For example, the data ecosystem 10 and most or all of its components may be behind an enterprise's firewall, and the data within the data ecosystem 10 is not transmitted over public networks or stored to public data storage systems.

The data sources 12 can be devices or systems that generate and/or accept data. For example, the data sources 12 could be devices or systems that include a camera (e.g., a video camera) such as a surveillance camera/system, smartphone, vehicle, drone, laptop, and the like. The data sources 12 could also include computer systems (e.g., hospital data storage systems, bank data storage systems, retail store data storage systems, personal computer terminals) to which data is entered manually or automatically. For example, a retail store data storage system may receive financial information from a customer's bank or creditor when a customer makes a purchase. The various data sources 12 can be physically located in different geographic regions (e.g., states, counties) that each have their own data laws.

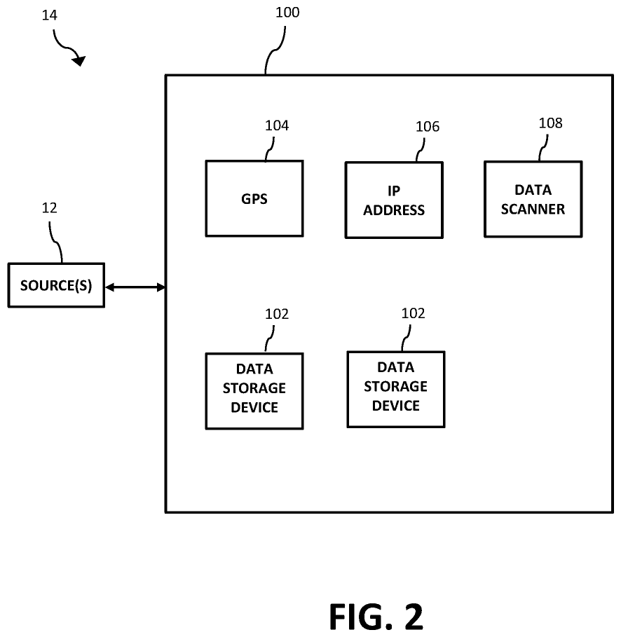

Although the data sources 12 themselves may have local data storage capabilities (e.g., memory), the local data storage is typically limited in data storage capacity and not designed for long-term data storage. As such, the data sources 12 may be communicatively coupled (e.g., wired or wirelessly) to one or more of the initial data storage systems 14 to offload data from time to time. Respective initial data storage systems 14 can be physically located in the same geographical locations as the respective data sources 12. Additional details of the initial data storage systems 14 are shown in FIG. 2 and discussed further below.

The initial data storage systems 14 can transmit the offloaded to data to the intermediate data storage systems 16, which can be considered to be longer-term data appliances compared to the initial data storage systems 14. For example, the intermediate data storage systems 16 can have larger storage capacities than the initial data storage systems 14 and may incorporate data storage techniques such as duplication, compression, and the like. In certain embodiments, the intermediate data storage systems 16 are physically located in the same geographic region as the data sources 12 and the initial data storage systems 14. In certain embodiments, the data sources 12 may be communicatively coupled to one or more of the intermediate data storage systems 16 without intervening initial data storage systems 14.

The intermediate data storage systems 16 can be communicatively coupled to the backup data storage systems 18A-C. The backup data storage systems 18A-C can each be physically located in different geographic locations from the intermediate data storage systems 16—with each geographic location potentially having their own data laws. As discussed in more detail below, the initial data storage systems 14 or the intermediate data storage systems 16 can check whether, based on characteristics of or metadata associated with the to-be-transferred data, the transfer of the data complies with local data laws and regulations before the data is transferred to one of the backup data storage systems 18A-C.

FIG. 2 shows a block diagram of an example of one of the initial data storage systems 14. In the example of FIG. 2, the initial data storage system 14 is a data shuttle 100—although other types of data storage systems can be used. The description and features of the data shuttle 100 provided below can apply to other types of initial data storage systems 14.

The data shuttle 100 includes one or more individual data storage devices 102 such as hard disk drives, solid state drives, and/or memory devices. Instructions for carrying out the various functions described below can be stored to one or more of the data storage devices 102. In certain embodiments, the initial data storage systems 14 include more than a single data shuttle 100. The initial data storage systems 14 may include a rack or other structure in which one or more of the data shuttles 100 can be physically inserted and removed. For example, when one of the data shuttles 100 has filled its available data storage capacity, the data shuttle 100 can be removed from the initial data storage system 14 and replaced with a new data shuttle. The removed data shuttle 100 can then be physically sent to a location with one of the intermediate data storage systems 16 and the data can be loaded to the intermediate data storage system 16. As another example, the initial data storage systems 14 can be communicatively coupled to a network (e.g., private network) and the data can be transmitted to one of the intermediate data storage systems 16 across the network.

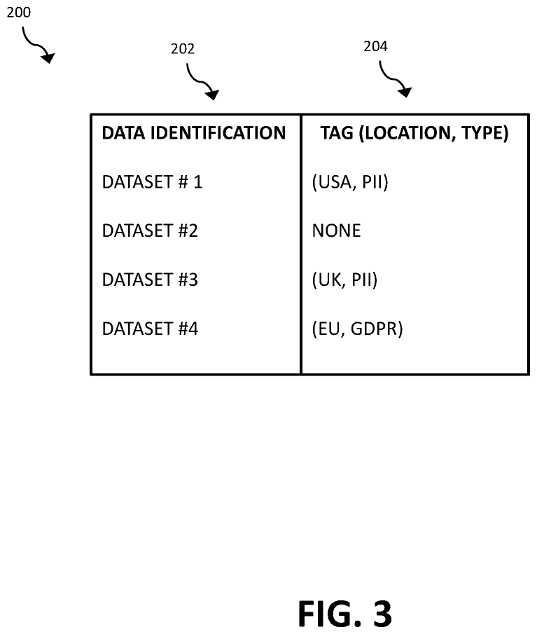

The data shuttle 100 can include a global positioning system (GPS) 104 and/or be assigned an internet protocol (IP) address 106. The GPS 104 and the IP address 106 can be associated with location data that indicates the geographic location of the data shuttle 100. After the data is transferred from the data sources 12 to the initial data storage system 14, the data can be associated with the location data from the GPS 104 or the IP address 106 by the initial data storage system 14. For example, the data can be tagged with the location of the initial data storage system 14. As will be described in more detail below with respect to FIG. 3, data structures can be used to maintain the data's association with the location data among other things.

The initial data storage system 14 can include a data scanner 108. As data from the data sources 12 is received by the initial data storage system 14, the data scanner 108 can determine whether the data is a type of data that is subject to data laws and regulations. For example, the data scanner 108 can be programmed or trained to automatically search for certain characteristics that indicate that the data is subject to data laws and regulations. These characteristics include certain formats for sensitive personal information such as social security numbers, home addresses, driver's license numbers, e-mail addresses, bank account numbers, and the like.

If the data scanner 108 detects data in the same format as, for example, a social security number, the data can be automatically determined to be subject to data laws and regulations and tagged as such. In certain embodiments, the data scanner 108 utilizes a library of data compliance indicators (e.g., predetermined formats indicating personally-identifiable information). The data compliance indicators stored in the library can be compared to the incoming data (e.g., scanned text) to help to determine whether the incoming data is subject to a data compliance law. The library of data compliance indicators can be stored in a database on one or more of the data storage devices 102 and can be updated from time to time. For example, the libraries stored among the fleet of initial data storage system 14 can be remotely updated to include additional types or formats of data compliance indicators.

If the data received by the initial data storage system 14 is determined to be subject to data laws and regulations, the initial data storage system 14 can associate the data with an indicator or tag that establishes that the data is subject to data laws and regulations. For example, the initial data storage system 14 can include a data tagger component 110 that generates metadata and associates datasets with certain metadata, which is described in more detail below. In certain embodiments, the data scanner 108 can also generate and associate datasets with metadata.

FIG. 3 shows an example data structure 200 that can be used to associate datasets with metadata that is relevant for managing where the datasets are transferred and stored. The data structure 200 includes data identification metadata 202 that associates a unique combination of alphanumeric characters with each separate dataset. The data structure 200 also includes tag metadata 204. In the example shown in FIG. 3, the tag metadata 204 includes location metadata and data type metadata. In certain embodiments, the data structure 200 only includes the tag metadata 204. Further, in certain embodiments, as the individual datasets are transferred to different data storage systems (e.g., the backup data storage systems 18A-C), the metadata associated with each dataset is maintained.

The location metadata can be generated by the GPS 104 and/or IP address 106 of the initial data storage system 14. The location metadata can include a unique combination of alphanumeric characters for each unique location. The location metadata indicates the geographic location of the data source 12 of the dataset. In the example of FIG. 3, the location metadata associated with dataset # 1 indicates that the dataset originated in the United States of America. In certain embodiments, the location metadata can indicate geographical regions that are more granular or less granular than country-by-country. For example, within the United States, individual states may have their own set of data laws and regulations, so the location metadata can indicate the individual state where the dataset originated.

The data type metadata can indicate the type of data contained in the respective datasets. In the example of FIG. 3, the data type metadata associated with dataset #1 indicates that the dataset includes personally-identifiable information or PII. As such, dataset # 1 originated in the United States and includes PII. In certain embodiments, datasets that do not include information that is subject to data laws and regulations do not include any metadata. Therefore, the datasets can first be scanned for certain types of data before the datasets are tagged with geographical metadata or other types of metadata.

After datasets are processed by the initial data storage system 14 (e.g., tagged with metadata), the datasets can be transferred to the intermediate data storage systems 16. As noted above, the data sources 12, the initial data storage systems 14, and the intermediate data systems 16 can be located in the same geographic location such that transferring the datasets between the data sources 12 and systems does not implicate data laws and regulations. As noted above, in certain embodiments, data sources 12 communicate data to the intermediate data storage systems 16 without first being communicated to the initial data storage system 14. As such, the features and functions described above with respect to the initial data storage system 14 can be contained and carried out by the intermediate data storage systems 16. Similarly, the initial data storage system 14 may communicate datasets to the backup data storage systems 18A-C without first being communicated to the intermediate data storage systems 16. As such, the initial data storage systems 14 may include the features and functions of the intermediate data storage systems 16 described below.

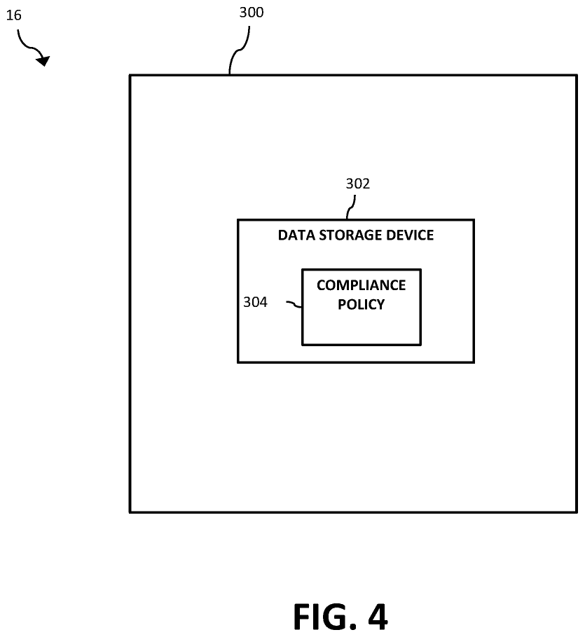

FIG. 4 shows a block diagram of an example of one of the intermediate data storage systems 16. In the example of FIG. 4, the initial data storage system 14 is a server 300—although other types of data storage systems can be used. The description and features of the server 300 provided below can apply to other types of intermediate data storage systems 16.

The server 300 can be located in a data center. Even when datasets are stored on servers 300 in a data center, it may be desirable to backup or duplicate certain datasets in a different geographic location. For example, in the event the data center loses power or connectivity, datasets that are backed up at a different geographic location can still be accessed. However, as noted above, different countries, states, etc., may have their own data laws and regulations that limit how and where data is transferred and stored.

The server 300 can include features that help to ensure that data transfers to the backup data storage systems 18A-C comply with applicable data laws and regulations. The server 300 can include data storage devices 302 such as hard disk drives, solid state drives, and/or memory. Instructions for carrying out the various functions described below can be stored to one or more of the data storage devices 302. In addition, the data storage devices 302 can store a compliance policy 304. The compliance policy 304 can include or represent applicable data rules and regulations of various geographic regions. For example, the compliance policy 304 may be a database of laws and regulations that can be referenced by components of the server 300. In certain embodiments, the compliance policy 304 includes at least some of the same information as the metadata of the data structure 200 described above. For example, the compliance policy can include a list of geographic regions, types of data, and locations where certain types of data cannot be transmitted or stored by geographic region.

The compliance policy 304 can be updated from time to time as different countries, etc., change or introduce new laws and regulations relating to data. For example, the compliance policies 304 stored across a fleet of intermediate data storage systems 16 can be remotely updated as data laws and regulations change.

After receiving datasets that have been tagged with metadata, the server 300 can determine whether and where to transmit and store a replica (e.g., duplicate copy, backup copy) of the datasets or the datasets themselves. In the data ecosystem 10 of FIG. 1, the server 300 can determine which of the backup data storage systems 18A-C are available to accept the datasets. The backup data storage systems 18A-C can each be located in different geographic regions. For example, the first backup data storage system 18A may be physically located in the United States, the second backup data storage system 18B may be physically located in Ireland, and the third backup data storage system 18C may be physically located in China.

When determining which of the backup data storage systems 18A-C can accept a given dataset, the server 300 can utilize the metadata associated with each dataset, the compliance policy 304, and geographic location data of the backup data storage systems 18A-C. In certain embodiments, such location data is determined based on the IP address of the backup data storage systems 18A-C.

The server 300 can select a first potential backup data storage system to receive and store the replica of the dataset. Before transmitting the replica of the dataset to the first potential backup data storage system, the server 300 can determine whether transmitting the replica to that potential backup data storage system will violate or comply with relevant data laws and regulations. For example, if (1) the metadata associated with the dataset indicates that the dataset originated in France and contains PII and (2) the location data of the first potential backup data storage system indicates China as the geographic location of the backup data storage system, the compliance policy 304 may dictate that the first potential backup data storage system is not available to receive the dataset.

If the first potential backup data storage system is determined to be a non-compliant destination for the dataset or a replica, a second potential backup data storage system can be selected, and the server 300 can carry out a similar process of utilizing the metadata of the dataset; the compliance policy; and the geographic location data of the second potential backup data storage system. The process of selecting potential backup data storage systems can continue until a compliant location is identified.

In addition to determining the final backup destination for the datasets or replicas thereof, the server 300 can make the same types of determinations for any additional intermediate data storage systems. For example, the datasets may need to pass through one or more other geographic locations before reaching their final destinations. Each of the intervening geographic locations can be analyzed to help ensure that data laws and regulations are complied with.

Once a compliant path and final destination are identified, the server 300 can transmit the datasets or their replicas along the path. In addition to transmitting the datasets or their replicas themselves, the metadata associated with the given datasets can be transmitted such that the geographic metadata and the data type metadata is not lost. As the datasets are further communicated or replicated, the metadata can be retained with the datasets. In certain embodiments, the metadata is retained, and the associations maintained during lifetime of the datasets. In certain embodiments, the various data storage systems include application program interfaces (APIs) that enable the metadata to be translated and used by the various data storage systems.

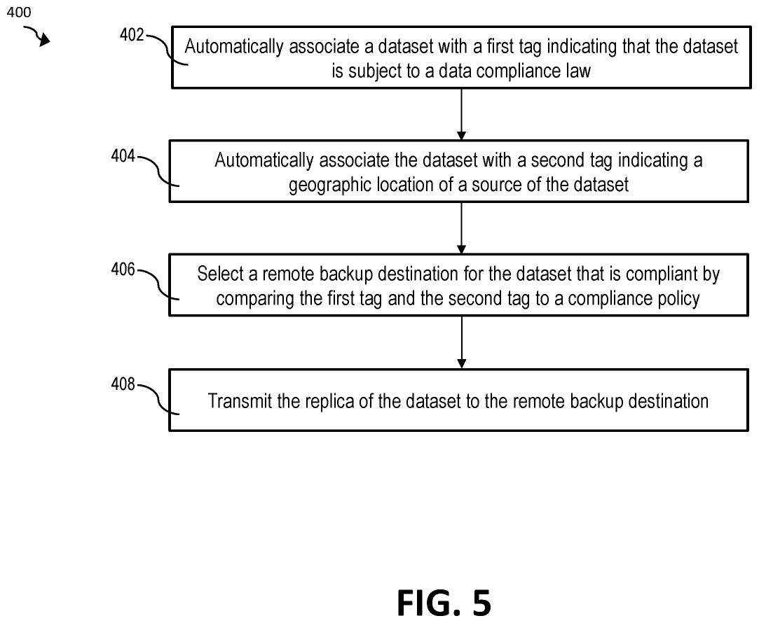

FIG. 5 shows a block diagram of a method 400 for transferring data between data storage systems. The various steps of the method 400 described below can be carried out in different orders and can be carried out in parallel and/or serially.

The method 400 includes automatically associating a dataset with a first tag indicating that the dataset is subject to a data compliance law (block 402 in FIG. 5). The method 400 further includes automatically associating the dataset with a second tag indicating a geographic location of a source of the dataset (block 404 in FIG. 5). The method 400 includes selecting a remote backup destination for the dataset that is compliant by comparing the first tag and the second tag to a compliance policy (block 406 in FIG. 5). The replica of the dataset is transmitted to the remote backup destination (block 408 in FIG. 5).

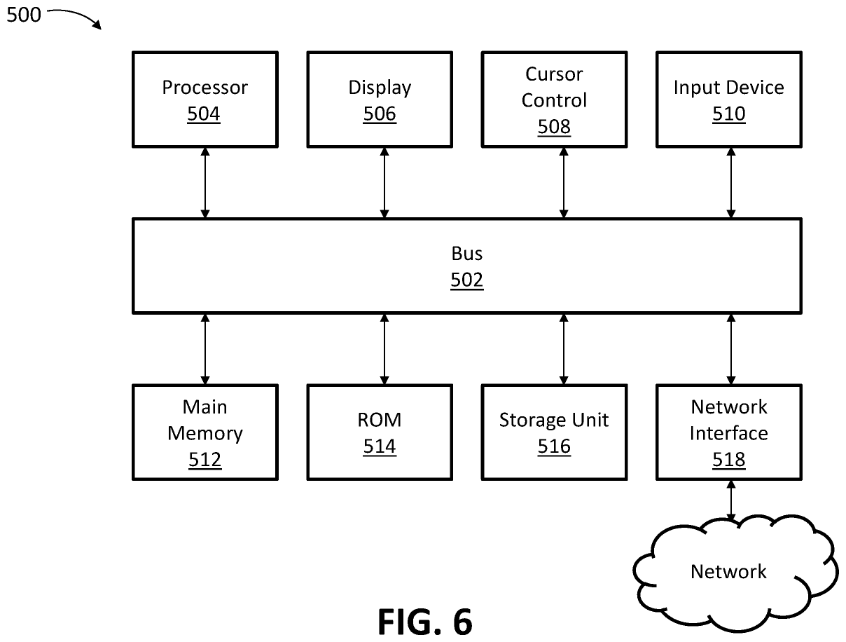

FIG. 6 shows a block diagram of illustrative components of a computer 500 for carrying out aspects of the functions and processes (e.g., the method 400) described above. For example, the initial data storage systems 14, the intermediate data storage systems 16, and the backup data storage systems 18A-C can each include one or more of their own computers 500.

The computer 500 can include a bus 502 or other communication mechanism for communicating information between or among a processor 504, a display 506, a cursor control component 508, an input device 510, a main memory 512, a read only memory (ROM) 514, a storage unit 516, and/or a network interface 518. In some examples, the bus 502 is coupled to the processor 504, the display 506, the cursor control component 508, the input device 510, the main memory 512, the ROM 514, the storage unit 516, and/or the network interface 518. And, in certain examples, the network interface 518 is coupled to a network.

In some examples, the processor 504 includes one or more general purpose microprocessors. In some examples, the main memory 512 (e.g., random access memory (RAM), cache and/or other dynamic storage devices) is configured to store information and instructions to be executed by the processor 504. In certain examples, the main memory 512 is configured to store temporary variables or other intermediate information during execution of instructions to be executed by processor 504. For example, the instructions, when stored in the storage unit 516 accessible to processor 504, render the computer 500 into a special-purpose machine that is customized to perform the operations specified in the instructions (e.g., the method 400 and other functions describe above). In some examples, the ROM 514 is configured to store static information and instructions for the processor 504. In certain examples, the storage unit 516 is configured to store information and instructions.

In some embodiments, the display 506 is configured to display information (e.g. alerts, status indicators) to a user of the computer 500. In some examples, the input device 510 (e.g., alphanumeric and other keys) is configured to communicate information and commands to the processor 504. For example, the cursor control 508 (e.g., a mouse, a trackball, or cursor direction keys) is configured to communicate additional information and commands (e.g., to control cursor movements on the display 506) to the processor 504.

Various modifications and additions can be made to the embodiments disclosed without departing from the scope of this disclosure. For example, while the embodiments described above refer to particular features, the scope of this disclosure also includes embodiments having different combinations of features and embodiments that do not include all of the described features. Accordingly, the scope of the present disclosure is intended to include all such alternatives, modifications, and variations as falling within the scope of the claims, together with all equivalents thereof.

I claim:

1. A method comprising:
automatically associating, via a computer, a dataset with a first tag indicating that the dataset is subject to a data compliance law;
automatically associating, via the computer, the dataset with a second tag indicating a geographic location of a source of the dataset;
selecting a remote backup destination for the dataset that is compliant by comparing the first tag and the second tag to a compliance policy; and
transmitting a replica of the dataset to the remote backup destination.

2. The method of claim 1, wherein the remote backup destination is a final destination for the dataset.

3. The method of claim 1, wherein transmitting the replica of the dataset further includes transmitting the first tag and the second tag along with the replica.

4. The method of claim 1, wherein the automatically associating the dataset with the first tag includes:
scanning text of the dataset,
comparing the scanned text to a library of data compliance indicators to determine whether the scanned text is subject to the data compliance law, and
associating the dataset with the first tag in response to the comparing the scanned text to the library.

5. The method of claim 4, wherein the library of data compliance indicators includes a library of data formats indicating personally-identifiable information.

6. The method of claim 1, wherein the automatically associating the dataset with the second tag indicating the geographic location includes:
determining the geographic location of the source based, at least in part, on an internet protocol address or a global positioning system, and
associating the dataset with the second tag in response to determining the geographic location.

7. The method of claim 1, wherein the selecting the remote backup destination includes:
selecting a first potential remote backup destination,
before transmitting the replica of the dataset to the first potential remote backup destination, determining that the first potential remote backup destination is not compliant with the compliance policy based on the first tag and the second tag,
selecting a second potential remote backup destination, and
determining that the second potential remote backup destination is compliant with the compliance policy, wherein the second potential remote backup destination is the remote backup destination.

8. A non-transitory computer-readable medium having stored thereon instructions that, when executed by a processor, cause the processor to:
automatically associate a dataset with a first tag indicating that the dataset is subject to a data compliance law;
automatically associate the dataset with a second tag indicating a geographic location of a source of the dataset;
select a remote backup destination for the dataset that is compliant by comparing the first tag and the second tag to a compliance policy; and
initiate transmission of a replica of the dataset to the remote backup destination.

9. The non-transitory computer-readable medium of claim 8, wherein transmitting the replica of the dataset further includes transmitting the first tag and the second tag along with the replica.

10. The non-transitory computer-readable medium of claim 8, wherein the automatically associating the dataset with the first tag includes:
scanning text of the dataset,
comparing the scanned text to a library of data compliance indicators to determine whether the scanned text is subject to the data compliance law, and
associating the dataset with the first tag in response to the comparing the scanned text to the library.

11. The non-transitory computer-readable medium of claim 8, wherein the automatically associating the dataset with the second tag indicating the geographic location includes:
determining the geographic location of the source based, at least in part, on an internet protocol address or a global positioning system, and
associating the dataset with the second tag in response to determining the geographic location.

12. The non-transitory computer-readable medium of claim 8, wherein the selecting the remote backup destination includes:
selecting a first potential remote backup destination,
before initiating transmission of the replica of the dataset to the first potential remote backup destination, determining that the first potential remote backup destination is not compliant with the compliance policy based on the first tag and the second tag,
selecting a second potential remote backup destination, and
determining that the second potential remote backup destination is compliant with the compliance policy, wherein the second potential remote backup destination is the remote backup destination.

13. A system comprising:
a first data storage system including a first computer configured to:
receive datasets from a data source,
associate the datasets with location metadata indicating the geographic origins of the datasets, based on a location from an internet protocol address or a global positioning system,
scan the datasets,
automatically determine that the dataset includes personally-identifiable information (PII) by comparing the scanned datasets to a library of data compliance indicators, and
associate the datasets with compliance metadata indicating that the datasets include PII.

14. The system of claim 13, further comprising:
a second data storage system including a second computer configured to:
receive the datasets, the location metadata, and the compliance metadata from the first data storage system, and
select a remote backup destination for the datasets that is compliant by comparing the location metadata and the compliance metadata to a compliance policy.

15. The system of claim 14, further comprising:
a backup data storage system located at the selected remote backup destination, wherein the second computer is configured to transmit replicas of the datasets to the remote backup destination.

16. The system of claim 15, wherein the second computer is configured to transmit the location metadata and the compliance metadata to the remote backup destination.

17. The system of claim 15, wherein the second computer is configured to retain a copy of the datasets, the location metadata, and the compliance metadata.

18. The system of claim 14, wherein the selecting the remote backup destination includes:
selecting a first potential remote backup destination,
before transmitting the datasets to the first potential remote backup destination, determining that the first potential remote backup destination is not compliant with the compliance policy based on the location metadata and the compliance metadata,
selecting a second potential remote backup destination, and
determining that the second potential remote backup destination is compliant with the compliance policy, wherein the second potential remote backup destination is the remote backup destination.

19. The system of claim 13, wherein the library of data compliance indicators includes a library of data formats indicating PII.

20. The system of claim 19, wherein the data formats include formats for social security numbers.
